(19) United States
(12) Patent Application Publication
(10) Pub. No.: US 2011/0055969 A1
(43) Pub. Date: Mar. 3, 2011

(54) Method to Produce Foreign Protein in Kernel of Oil Palm

(75) Inventor: Ahmad Parveez GHULAM KADIR, Bandar Baru Bangi (MY)

(73) Assignee: Malaysian Palm Oil Board

(21) Appl. No.: 12/874,170

(22) Filed: Sep. 1, 2010

(30) Foreign Application Priority Data
Sep. 1, 2009 (MY) PI 20093620

(51) Int. Cl. C12N 15/82

(52) U.S. Cl. 800/281; 800/278; 800/287

(57) ABSTRACT

The present disclosure relates at least in part to methods for use in producing a foreign protein in a plant, for example an oil palm. In certain embodiments the foreign protein is expressed in the kernel of an oil palm fruit.

### CROSS-REFERENCE TO RELATED APPLICATIONS

The present application claims priority to Malaysian Patent Application No. PI 20093620, titled Method to Produce Foreign Protein in Kernel of Oil Palm, filed Sep. 1, 2009, which is incorporated herein by reference in its entirety including all drawings.

### FIELD OF INVENTION

The present disclosure relates at least in part to transgenic plants and methods of making thereof, as well as expression of proteins in plants and/or plant tissues.

### BACKGROUND OF THE INVENTION

The following description is provided simply as an aid in understanding the invention and is not admitted to describe or constitute prior art.

Huge gains can be obtained based on the possibility and understanding of altering and transferring genes from one plant to another, with the assistance of scientific tools. In many cases, the primary objective of plant gene manipulation in most cases is to provide significant improvement of quality and more particularly for increasing total yield of the respective crop or plant.

Proceeding from the above, one of the most important perennial crops amenable for such technological solutions with respect to the production of excellent quality products is the oil palm (E. guineensis), whereby the genetic efforts are typically focused on two main tissues of the oil palm, namely the mesocarp and kernel.

### SUMMARY OF INVENTION

The present invention is directed to a method for use in producing foreign protein in the oil palm, and more particularly in the kernel of oil palm fruit.

In a first aspect provided are methods for use in producing foreign protein in the kernel tissues of an oil palm fruit. The methods may include one or more of the following: (a) preparing explants (e.g., young unopened leaves, immature embryos and/or roots) from an oil palm plant; (b) preparing callus and followed by embryogenic callus from the prepared oil palm explant; (c) bombarding a foreign gene mixture into a oil palm embryogenic callus, prepared for example using steps (a-b); and/or producing the bombarded embryogenic callus in the form of polyembryogenic cultures; (d) re-generating said bombarded embryogenic callus into a whole transgenic plant; and (e) isolating the seed from the transgenic plant fruit and preparing the seed for germination; (f) confirming the presence of the gene in the plant and/or (g) proving the presence of gene product in the kernel of the plant fruits.

In another aspect, provided are methods for producing a foreign gene product or protein in the tissues of transgenic oil palm plant. The methods may include one or more of the following: (a) preparing an explant from an oil palm plant; (b) preparing callus and followed by embryogenic callus from said prepared oil palm explant; (c) bombarding a foreign gene mixture into the oil palm embryogenic callus, prepared, for example, using steps (a-b); (d) selecting transformed said embryogenic callus on selection agents; (e) proliferating and regenerating the selected embryogenic callus into a whole plant thus producing transgenic plant; (f) confirming the presence and expression of the foreign gene in the plant; and/or (g) proving the presence of foreign gene product or foreign protein in the kernel of the plant fruits.

In some embodiments of the methods provided herein, the methods further include the step of confirming the presence of the foreign gene product or foreign protein in a section or portion of the transgenic plant. In certain embodiments a method as provided is used in producing a foreign gene product or foreign protein in the kernel of oil palm fruit. In various embodiments, the expression of foreign genes and production of foreign gene product or protein is observed in the kernel section of oil plant fruit. In some embodiments the explant may include young unopened leaves, immature embryos and/or roots. In some embodiments, the production of foreign gene product or foreign protein in the kernel section of oil palm fruits is directed by driving the foreign gene with a promoter which is specific to kernel of oil palm. In various embodiments of any of the methods provided herein, the transgenic oil palm plant exhibits increased yield of kernel oil, modified lipids and/or non-lipid components of palm kernel oil; and/or improved quality palm kernel oil, production of industrial oils and/or chemicals and nutraceuticals and pharmaceutical compounds. In some embodiments of any of the methods provided herein, the foreign gene includes one or more genes selected from the group consisting of a gene encoding one or more of acetyl CoA carboxylase (ACCase), β-ketoacyl ACP synthase II (KASII), ketoacyl ACP synthase I (KASI), ketoacyl ACP synthase III (KASIII), palmitoyl ACP thioesterase and other thioesterases, stearoyl ACP desaturase and other desaturases, oleoyl CoA desaturase, fatty acid elongases, oleate hydroxylase, acyltransferases, β-ketothiolase, threonine deaminase/dehydratase, acetoacetyl CoA reductase and/or polyhydroxybutyrate synthase.

As used herein, the term “about” in quantitative terms refers to plus or minus 10%. For example, “about 3%” would encompass 2.7-3.3% and “about 10%” would encompass 9-11%. Moreover, where “about” is used herein in conjunction with a quantitative term it is understood that in addition to the value plus or minus 10%, the exact value of the quantitative term is also contemplated and described. For example, the term “about 3%” expressly contemplates, describes and includes exactly 3%.

### DETAILED DESCRIPTION

In line with the above summary, the disclosed description and examples relate in part to methods for use in producing a foreign protein in a plant, and in some embodiments in the kernel of oil palm fruit, however it shall be apparent to one skilled in the art that the exemplifications are provided to better elucidate various embodiments of the present inventions and therefore should not be construed as limiting the scope of protection.

Manipulation of plant genes to obtain valuable engineered products is an important aspect of plant production. Often, foreign DNA may isolated and introduced into host cells, via transformation of cells. Each of these steps may be performed via several methods, subject to the variety and characteristics of the respective plant.

The current disclosures may in some embodiments provide methods for regulating characteristics in plants and in some embodiments address certain shortcomings of conventional breeding. In some aspects of the methods disclosed herein, biolistics (biological ballistic)-mediated transformation method is provided for the production of transgenic oil palm.

Accordingly, in various embodiments of the present disclosure, provided are methods for use in producing foreign protein in oil palm, and more particularly in the kernel part of oil palm fruit.

In some embodiments the provided methods relate to the regeneration of transgenic oil palm using a biolistics-mediated approach.

Generally the pericarp of the oil palm fruit comprises three layers, the exocarp (skin), epicarp (mesocarp—outer pulp containing palm oil in fibrous matrix form) and a kernel (the endosperm) which contains oil and carbohydrate reserves for the embryo and a part known as the endocarp for enclosing the kernel. The mesocarp of all fruit contains fibres which run longitudinally through the oil bearing tissue. The said fibrous material typically constitutes about 16% of the mesocarp weight but may vary from 11 to 21%. Further, relevant studies have shown that the oil content of the mesocarp of ripe fruit varies from under 40% to over 60%. It is therefore understood by a person skilled in the art that palm oil is extracted from the mesocarp section, and the palm kernel oil is extracted from the kernel section.

The present disclosure in some embodiments provides a biolistics-mediated approach with respect to transferring the Biolistic (biological ballistic) as an alternative technical approach in transfecting cells, in which with the methods involve bombarding the cells with particles containing DNA. In order to elevate efficiency, optimization of biological and physical parameters affecting DNA delivery into oil palm embryogenic calli, selection of suitable promoters and evaluation for effective selection agents may be carried out. With the optimized parameters, bombarded embryogenic calli may be exposed to herbicide Basta (active ingredients glufosinate ammonium) until resistant embryogenic calli are obtained.

In some aspects and embodiments of the present methods, transgenic embryogenic calli may be regenerated into whole plants. The transgenic oil palm status may be accordingly verified by molecular and protein analyses. Subsequently, the verified palms may be grown to maturity and preferably be proven to be fertile. Expression of foreign genes and production of its protein may occur in and may be observed in the kernel of oil palm fruit.

In certain embodiments of the disclosed methods, a Biolistics PDS-1000/He(Bio-Rad) apparatus is used to deliver DNA into oil palm embryogenic calli. The oil palm embryogenic calli may be bombarded with a plasmid, for example, a pAHC25 plasmid (a plasmid carrying bar and gusA genes both under the control of Ubiquitin 1 promoter) (Christiensen et. al 1992).

The following are examples, which illustrate procedures for practicing the invention. These examples should not be construed as limiting.

### EXAMPLES

### Example I

### Callus Initiation from Oil Palm Leaflet, Roots and Immature Embryos

Leaflets from unopened (−6) frond and in vitro roots were aseptically transferred onto solid callus initiation medium [(MS salts (Murashige and Skoog, 1962)+Y3 vitamins (Eeuwans, 1976)+0.1 g/1 myo-Inositol and L-glutamin+3% sucrose+5×10⁻⁵M, 2, 4-D+0.25% activated charcoal+0.7% agar] and incubated at 28° C. in the dark. Any callus formed was subcultured every four weeks onto the same medium until embryogenic calli were formed.

Immature embryos were collected 15 weeks after anthesis. After sterilization, callus was initiated on the following media (Y₃ macro, micro nutrient and vitamin, +0.05% (w/v) cystein+0.5% (w/v) polyvinyl pyrolidone (PVP₄₀)+0.3% (w/v) activated charcoal+5×10⁻⁴M 2,4-D+0.22% (w/v) gelrite {Gibco-BRL}) according to Teixera et al., (1993) and incubated at 28° C. in the dark. Any callus formed was subcultured every four weeks onto the same medium until embryogenic calli were formed.

### Example II

### Maintenance of Embryogenic Calli

The step of the above Example I was for the preparation of the embryogenic callus of the oil palm for use in the bombardment process. The callus was maintained on agar medium furnished with suitable MS macro and micronutrients prior to being subcultured every 30 days onto fresh medium. It is understood that the steps used or exemplified herein for preparation of the callus may vary and any of such variations may be suitable for the presently provided methods.

The embryogenic callus was maintained on agar-solidified medium containing MS macro and micronutrients supplemented with 2.2 mg/l 2,4-D and 30 gm/l sucrose. The medium was adjusted to pH 5.7 with KOH prior to autoclaving. Embryogenic callus was cultured at 28° C., in the dark, and subcultured every 30 days onto fresh medium.

### Example II

### Bombardment of Embryogenic Calli

The next step was the bombardment of embryogenic calli, which may be carried out based on standard procedures. The main objective is to transfer the selected DNA involved for producing foreign protein into the tissue of oil palm to be expressed and thus reproduction of said protein.

In this step, five microlitres of DNA solution (1 μg/μl), 50 μl of CaCl2 (2.5M) and 20 μl spermidine (0.1M free base form) were added sequentially to the 50 μl particles suspension. The mixture was vortexed for 3 minutes, spun for 10 seconds at 10,000 rpm and the supernatant discarded. The pellet was washed with 250 μl of absolute ethanol. The final pellet was resuspended in 60 μl of absolute ethanol. Six microlitres of the solution was loaded onto the centre of the macrocarrier and was air dried. Bombardments were carried out once at the following conditions; 1100 psi rupture disc pressure; 6 mm rupture disc to microcarrier distance; 11 mm microcarrier to stopping plate distance, 75 mm stopping plate to target issue distance and 67.5 mmHg vacuum pressure.

### Example IV

### Production of Oil Palm Polyembryogenic Cultures

The production of cultures obtained from the previous step may be carried out with a standard procedure and not confined to the steps as described below.

Embryogenic cultures were maintained on media containing MS macro and micronutrients and Y3 vitamins supplemented with 100 mg/l each of myo-inositol, L, glutamine, L-arginine and L-asparagine, 5 μM IBA, 0.7% agar and 30 gm/l sucrose to form polyembryogenic cultures. The medium was adjusted to pH 5.7 with KOH prior to autoclaving. Embryogenic calli were incubated at 28° C. in the presence of light and were subcultured every 30 days onto fresh medium.

### Example V

### Small Plantlets Production from Polyembryogenic Cultures

The subsequent step was to produce small plantlets based on the polyembryogenic cultures prepared in the previous steps. It can be carried out based on known standards or procedures, an exemplary of a method is as described below:

The small plantlets were produced from polyembryogenic cultures on media containing MS macro and micronutrients and Y₃ vitamins supplemented with 100 mg/l each of myo-inositol, L-glutamine, L-arginine and L-asparagine, 0.1μ NAA, 0.4% agar and 30 gm/l sucrose. The medium was adjusted to pH 5.7 with KOH prior to autoclaving. Polyembryogenic calli were incubated at 28° C. in light until sufficient shoots were produced.

### Example VI

### Root Initiation from Oil Palm Cultures

Roots initiation were carried out based on the small plantlets on media containing MS macro and micronutrients and Y₃ vitamins supplemented with 300 mg/l L-glutamine, 100 mg/l myo-inositol, 10 μM 2,4-D, 70 μM NAA, 0.15% activated charcoal and 60 gm/l sucrose. The medium was adjusted to pH 5.7 with KOH prior to autoclaving. These plantlets were incubated at 28° C. in light until roots formed. The full regenerated plantlets were preferably transferred into polybags in the next step and accordingly grown in a biosafety screen house.

### Example VII

### Planting of Transgenic Oil Palm

As for the planting of transgenic oil palm, the plantlets were grown in biosafety screen house in polybags. The plantlets were fertilized and treated with insecticide using normal nursery standard. After six months, the plantlets were transferred into a bigger polybag. The transfer into a bigger polybag was carried out several times until the plant arrived to maturity, flowering and producing fruits.

### Example VIII

### Transmission of Transgenes in Progenies

The transmission of transgenes in progenies may involve preparing the seeds from the transgenic plants, whereby said seeds from fruit or fruit from non-transgenic oil palm pollinated with transgenic pollen are cleaned and transferred onto soil. Once the seed was germinated, and eventually produced seedlings, DNA was isolated from these plants and was subjected to PCR analysis to detect the presence of transgenes.

### Example IX

### GUS Histochemical Assay on Kernel Slices

The activity of the promoter of the protein in the plant tissue, in this case the kernel slices can be determined based on a standard procedure, for instance the GUS histochemical assay. For this assay, fruits were sterilized with Sodium Hypochlorite and ethanol followed by slicing the fleshy tissue into thin slices. These slices were later subjected to GUS histochemical staining. However, as the kernel tissues are rich with oil, the tissue slices were fixed for 5 minutes on ice in fixation solution containing 5% formaldehyde in sodium phosphate buffer pH 7.0. GUS assay buffer (0.1M NaPO₄ buffer, pH 7.0, 0.5 mM K-Ferricyanide, 0.5 mM K-Ferrocyanide, 0.01M EDTA, 1 mg/ml 5-Bromo-4-Chloro-3-Indolyl-13-D-glucuronide acid (dissolved in Dimethyl Formamide at 50 mg/ml) and 1 μl/ml Triton X-100)(Klein et at., 1988)+20% v/v methanol was filter sterilized and stored at −20° C. in the dark. The tissues were stained overnight (20 hours) with GUS buffer at 37° C., and stained tissues were scored optically using a Nikon SMZ-U stereoscopic zoom microscope and photographed with the NIKON UFX-DX system.

### Example X

### Preparation of Total DNA from Embryoids and Small Plantlets

Resistant embryoids and leaflets were selected randomly and subjected to total DNA isolation, carried out according to the method of Ellis (1993). Tissues (10-50 mg) were placed in a 1.5 ml microfuge tube and immersed in liquid nitrogen. Frozen tissues were ground to a fine powder in the presence of 400 μl EB2 buffer (500 mM NaCl, 100 mM Tris-Cl [pH 8.0] and 20 μl 20% SDS. Four hundred μl of phenol mix (1:1; phenol: chloroform) were then added, thoroughly mixed and centrifuged (12,000 rpm, 2 min, RT). The aqueous phase was transferred to a new tube and mixed with 800 μl absolute ethanol. DNA was recovered by centrifugation (12,000 rpm, 5 minutes, RT). The pellet was washed with 70% ethanol and dissolved in 50 μl TE buffer (10 mM Tris-Cl and 1 mM EDTA, pH 8.0).

### Example XI

### Polymerase Chain Reaction (PCR)

Amplification of transgenes can be carried out using standard and touch down PCR protocols (Sambrook et. al 1989). 50 ng of oil palm DNA and one ng of transforming plasmid DNA were used in PCR reactions. In the standard procedure the following condition was used: 30 cycles at 92° C. (50 sec), 60° C. (50 sec) and 72° C. (60 sec). For the touch down procedure, 10 cycles 92° C. (45 sec), 70° C. (45 sec-0.5° C. per cycle), 72° C. (60 sec) and 20 cycles 92° C. (45 sec), 65° C. (45 sec) and 72° C. (60 sec) was used. Amplified DNA fragments were checked by electrophoresis on 1.4% agarose gels in 0.5×TBE (45 mM Tris-Borate; 1 mM EDTA, pH 8.0) buffer.

### Example XII

### Southern Blot Hybridization Analysis

It would be apparent to a person skilled in the art that the Southern blot hybridization analysis is a standard methodology and thereby the steps involved in this regard is based on the method of Southern (1975). Accordingly, digested DNA from 0.8% agarose gel in 1×TBE buffer was capillary transferred onto nylon membrane. Agarose gel containing 10 μg of the BamH1-digested DNA was soaked in depurinating buffer (0.2 M HCl) for 10 minutes, denaturing buffer (1.5M NaCl and 0.5M NaOH) for 45 minutes and transferred into neutralization buffer (1M Tris pH 8.0 and 1.5 M NaCl) for 1 hour. The gel was later transferred onto 3 mM paper with the end of the paper, paper towels, a glass plate and a 500 g weight. The set up was left overnight (16-20 hours) in order to allow al the DNA to be transferred onto the membrane. After transfer, the membrane was washed with 2×SSC and baked at 80° C. for 2-4 hours.

It is noted that the oligolabeling of the bar gene fragment (prepared by PCR or fragment isolated from gel after restriction digestion of the transforming plasmid, pAHC25) for use as probe was carried using the method as provided by Feinberg and Volstein (1983). Using this method, DNA (6 μl˜10 ng) was added to 20 μl 5×OLB (0.25 M Tris-HCl pH 8.0, 25 mM MgCl₂ 0.36% v/v 2-mercaptoethanol, 1 M hepes pH 6.6, 30% hexadeoxyribonucleotides (90 O.D. units/ml)), boiled for 5 minutes and chilled on ice. The following was added to the above mixture: 2 μl 0.1M dNTPs (except dCTP), 5 μl α³² P (dCTP) 370 KBq/μl, 2 μl 10 mg/ml BSA, 1 μl Klenow (6 U/μl) and 14 μl distilled water. The labeling reaction was carried out by incubating at 37° C. for 30 minutes. Probe was denatured by the addition of 50 μl 1 M HCl for 1 minute and 50 μl Tris-HCl (pH 7.5) for 1 minute. The denatured probe was stored on ice until use.

Pre-hybridization and hybridization were carried out using the same buffer. Membrane, transferred with DNA, was pre-hybridized in pre-hybridization buffer (40% pipes/NaCl pH 6.8 (1.5% pipes, 8.7% NaCl and 0.37% EDTA [pH 8.0]); 20% Denhardts 50×[1% BSA, 1% Fieoll, 1% PVP and 10% SDS]; 0.5% SS-DNA 1-mg/ml) and 39.5% distilled water) for 90 minutes at 65° C. Denatured DNA probe was added and hybridized for 20 hours at 65° C. After hybridization, the membrane was prewashed once with 2×SSC for 1 minute and washed twice in 0.1×SSC and 0.1% w/v SDS. The first wash carried out for 30 minutes and the second one for 45 minutes, both at 65° C. The washed membrane was wrapped with saran wrap and exposed to X-ray film with an intensifying screen at −70° C.

It is noted from the method as discussed above (relating to certain embodiments of the methods provided herein) the DNA was successfully delivered into oil palm embryogenic calli. It is mentioned that the oil palm embryogenic calli were bombarded with plasmid pAHC25—a plasmid carrying bar and gusA genes both under the control of Ubiquitin 1 promoter (Christiensen ct.al., 1992). It should be mentioned that Bar gene will give resistant to herbicide Basta and gusA gene will give a blue pigmentation after addition of a substrate, 5-Bromo-4-Chloro-3-Indolyl-β-D-glucuronide acid.

It is observed that tissue bombarded with microcarriers lacked DNA as well as non-bombarded tissues did not show transient gusA gene expression. Optimization was carried out using transient gusA gene expression as an indicator of the efficiency of the parameters studied. Each blue spot that arises from the histochemical localization of GUS activity, whether in a single cell or a group of cells, was considered or indicates as one expression unit, as defined by Klein et al. (1988).

Further, transgenic oil palm embryogenic calli were selected on the selection medium provided with herbicide Basta at the preferred concentration of 40 to 80 mg/l one week after bombardment. Bombarded embryogenic calli were cultured on medium free of the selective agent for one week. It is further observed that the untransformed cells began to die and that only resistant cells proliferated.

The regeneration of transgenic oil palm plants using some embodiments of the methods provided herein may involve a main step of transferring the embryogenic callus onto fresh polyembryogenesis inducing medium, with a selective agent. Another alternative was to conduct a step-by-step selection. In this process, an initial selection was carried out by way of exposing the bombarded embryogenic callus to half strength selective agent (20 mg/l). It is noted that the selected transgenic embryogenic callus was then subcultured onto medium containing full strength of the selective agents (40 mg/l) after one month. After 2-3 subcultures, the transgenic embyrogenic callus was then transferred onto a polyembryogenic induction medium for regeneration of said callus.

It can be seen that whitish and greenish polyembryogenic callus started to develop after three to five months of culture on polyembryogenesis inducing medium. It is further observed that after several months, the polyembryogenic cultures started to produce shoots. They were subsequently isolated individually and transferred onto shooting medium for shooting elongation. In this process, the shoots were accommodated by test tubes containing liquid medium for further development and root initiation.

The molecular and protein analyses of transgenic plants involve the isolation of DNA from a few plantlets and later on subjected to PCR, prior to Southern Hybridization analyses. On the other hand, DNA from untransformed plants was as well isolated and thus used as negative controls. DNA which was isolated from transgenic embryogenic callus was also used as a positive transformed control. DNA concentration and purity were determined by using conventional means, particularly Bio-Rad Protein Assay Kit. The concentration of protein was calculated to ensure that the equal amounts of protein were used for analysis.

In another step, DNA from the putative transformed plants and one untransformed plant was subjected to amplification of an oil palm internal control sequence for reliable PCR analysis of the transgene. It is observed that the transformed plants showed the amplification of the bar gene used for selection. In addition, no amplification of the bar gene was observed for the untransformed control and the water control.

As for the Southern Blot hybridization Analysis, the hybridization process was accordingly carried out on undigested DNA isolated from at least five regenerated plants derived from a different resistant embryogenic callus clumps and form one untransformed control plant. In this experiment, a PCR amplified bar gene or fragment isolated from gel after restriction digestion of the transforming plasmid, pAHC25was used as probe for the hybridization. Hybridization to undigested high molecular weight DNA was observed with all the regenerants and the positive control. As for the untransformed control, no hybridization was observed. It is further noted that the hybridization to high molecular weight DNA indicated that stable integration of the transgene into the genome of regenerated oil palm plants has occurred.

In order to prove that the introduced gene is functional, the phosphinothricin acetyltransferase analysis was performed. The presence of an active bar gene was confirmed by determining the product of the gene, said product is an enzyme known as phosphinothricin acetyltransferase (PAT). The enzyme functions mainly to inactivate phosphinothricin (an active ingredient of herbicide Basta) by acetylation (DeBlock et al., 1987). It is observed that protein isolated from Basta resistant transgenic plants has demonstrated PAT activity based on thin layer chromatography analysis while the protein isolated from untransformed plant did not show any PAT activity.

The planting of transgenic plantlets may involve the steps of transferring plantlets in polybags and thus left for growth for at least two years. These plants are placed in a fully contained biosafety screen house and covered with size 50 mesh and fixed with double layer door to trap and prevent insects and small animals from entering the screen house during opening and closing of the same. The plants later started flowering and the flowers were bagged to avoid escape of pollens. It is further observed that these plants eventually produce female flowers fruits after controlled pollination, fruits from these plants were selected and the seeds were germinated to produce T1 progenies. PCR analysis was conducted on these plants and results have indicated the presence of bar gene.

The final step is to provide GUS analysis on transgenic oil palm fruits. In this step, the kernel slices were accordingly subjected to histochemical gusA gene assay. Results based on the assay indicate that the gene is fully functional by synthesizing the foreign protein or enzyme in the kernel of oil palm fruit.

With certain embodiments of the methods provided herein, a transgenic oil palm plant can be obtained, the oil palm plant was observed to exhibit the following characteristics; increased oil yield, modified lipids and non-lipid components of palm oil and improved quality palm oil, production of industrial oils and chemicals and nutraceuticals and pharmaceutical compounds.

In another aspect, the transgenic plant obtained based on the preferred embodiments of the present methods utilizes genetic material that encodes acetyl CoA carboxylase (ACCase), β-ketoacyl ACP synthase II (KASII), ketoacyl ACP synthase I (KASI), ketoacyl ACP synthase III (KASIII), palmitoyl ACP thioesterase and other thioesterases, stearoyl ACP desaturase and other desaturases, oleoyl CoA desaturase, fatty acid elongases, oleate hydroxylase, acyltransferases, β-ketothiolase, threonine deaminase/dehydratase, acetoacetyl CoA reductase and/or polyhydroxybutyrate synthase.

It is understood by a person skilled in the art that the methods for experiments and studies are described as exemplifications herein and thus the results are not intended, however, to limit or restrict the scope of the invention in any way and should not be construed as providing conditions, parameters, agents or starting materials which must be utilized exclusively in order to practice the present invention. It is therefore understood that the invention may be practiced, within the scope of the appended claims, with equivalent methods for the experiments than as specifically described and stated in claims.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the invention claimed.

Thus, it should be understood that although the present invention has been specifically disclosed by preferred embodiments and optional features, modification, improvement, and variation of the inventions disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

1. A method for use in producing genetically-modified oil palm plant comprising the steps of;
(a) preparing an explant from an oil palm plant;
(b) preparing callus and followed by embryogenic callus from said prepared oil palm explant;
(c) bombarding a foreign gene mixture into the oil palm embryogenic callus of steps (a-b);
(d) selecting transformed said embryogenic callus on selection agents;
(e) proliferating and regenerating the selected embryogenic callus into a whole plant thus producing transgenic plant;
(f) confirming the presence and expression of the foreign gene in said plant; and
(g) proving the presence of foreign gene product or foreign protein in the kernel of the said plant fruits.

2. The method as claimed in claim 1 wherein the method is used in producing a foreign gene product or foreign protein in the kernel of oil palm fruit.

3. The method of claim 1, wherein the expression of foreign genes and production of foreign gene product or protein is observed in the kernel section of oil plant fruit.

4. The method of claim 1 wherein the explant comprises young unopened leaves, immature embryos and/or roots.

5. The method of claim 1 wherein the method is for use in producing foreign gene product or foreign protein is in the kernel of oil palm fruit.

6. The method of claim 1 wherein the production of the foreign gene product or foreign protein in the kernel section of oil palm fruits is directed by driving the foreign gene with a promoter which is specific to kernel of oil palm.

7. The method of claim 1 wherein said transgenic oil palm plant exhibits increased yield of kernel oil, modified lipids and/or non-lipid components of palm kernel oil and/or improved quality palm kernel oil, production of industrial oils and/or chemicals and/or nutraceutical and/or pharmaceutical compounds.

8. The method claim 1 wherein the foreign gene encodes acetyl CoA carboxylase (ACCase), β-ketoacyl ACP synthase II (KASH), ketoacyl ACP synthase I (KASI), ketoacyl ACP synthase III (KASIII), palrnitoyl ACP thioesterase and other thioesterases, stearoyl ACP desaturase and other desaturases, oleoyl CoA desaturase, fatty acid elongases, oleate hydroxylase, acyltransferases, β-ketothiolase, threonine deaminase/dehydratase, acetoacetyl CoA reductase and/or polyhydroxybutyrate synthase.

9. A method for use in producing of foreign gene product or protein in the tissues of transgenic oil palm plant comprising the steps of:
(a) preparing an explant from an oil palm plant;
(b) preparing callus and followed by embryogenic callus from said prepared oil palm explant;
(c) bombarding a foreign gene mixture into the oil palm embryogenic callus of steps (a-b);
(d) selecting transformed said embryogenic callus on selection agents;
(e) proliferating and regenerating the selected embryogenic callus into a whole plant thus producing transgenic plant;
(f) confirming the presence and expression of the foreign gene in said plant; and
(g) proving the presence of foreign gene product or foreign protein in the kernel of the said plant fruits.

10. The method of claim 9 wherein the method is used in producing foreign gene product or foreign protein in the kernel of oil palm fruit.

11. The method of claim 9 wherein the expression of foreign genes and production of novel protein is observed in the kernel section of oil plant fruit.

12. The method of claim claim 9 wherein the explant comprises young unopened leaves, immature embryos and/or roots.

13. The method of claim 9 wherein the method is for use in producing foreign gene product or foreign protein in the kernel of oil palm fruit.

14. The method of claim 9 wherein the production of the foreign gene product or foreign protein in the kernel section of oil palm fruits is directed by driving the foreign gene with a promoter which is specific to kernel of oil palm.

15. method of claim 9 wherein said transgenic oil palm plant exhibits increased yield of kernel oil, modified lipids and/or non-lipid components of palm kernel oil and/or improved quality palm kernel oil, production of industrial oils and/or chemicals and/or nutraceutical and/or pharmaceutical compounds.

16. The method of claim 9 wherein the foreign gene encodes acetyl CoA carboxylase (ACCase), β-ketoacyl ACP synthase II (KASII), ketoacyl ACP synthase I (KASI), ketoacyl ACP synthase III (KASIII), palmitoyl ACP thioesterase and other thioesterases, stearoyl ACP desaturase and other desaturases, oleoyl CoA desaturase, fatty acid elongases, oleate hydroxylase, acyltransferases, β-ketothiolase, threonine deaminase/dehydratase, acetoacetyl CoA reductase and/or polyhydroxybutyrate synthase.
